(19) United States
(12) Patent Application Publication
(10) Pub. No.: US 2026/0137344 A1
(43) Pub. Date: May 21, 2026

(54) WEARABLE DEVICE

(71) Applicant: STMicroelectronics International N.V., Geneva (CH)

(72) Inventors: Arthur ARNAUD, La Tronche (FR); Jonathan STECKEL, Corenc (FR)

(73) Assignee: STMicroelectronics International N.V., Geneva (CH)

(21) Appl. No.: 19/395,030

(22) Filed: Nov. 20, 2025

(30) Foreign Application Priority Data
Nov. 20, 2024 (FR) FR2412681

(51) Int. Cl. A61B 5/00; G04G 21/02

(52) U.S. Cl. CPC A61B 5/681; A61B 5/0059; G04G 21/025

(57) ABSTRACT

A wearable device includes an image sensor having a matrix of pixels. Each pixel includes at least one light emitter and at least one photosensor. A first group of pixels of the matrix includes one light emitter configured to emit wavelengths selected among near and short-wave infrared wavelengths. A second group of pixels of the matrix includes a photosensor sensitive to the wavelengths selected among near and short-wave infrared wavelengths.

### PRIORITY CLAIM

This application claims the priority benefit of French Application for Patent No. FR2412681, filed on November 20, 2024, the content of which is hereby incorporated by reference in its entirety to the maximum extent allowable by law.

### TECHNICAL FIELD

The present disclosure relates generally to wearable devices and their operating method.

### BACKGROUND

Some wearable devices, such as smartwatches, carry a sensor to monitor body vital signs.

These devices are not accurate and are limited regarding their ability to detect different diseases or physiological parameters.

There is a need to provide a wearable device which can measure, more accurately, a wider range of physiological parameters while also be capable of biometrical authentication.

### SUMMARY

One embodiment addresses all or some of the drawbacks of known wearable devices.

One embodiment provides a wearable device comprising an image sensor having a matrix of pixels, each pixel comprising at least one light emitter and at least one photosensor; at least one first group of pixels of the matrix comprising one light emitter configured to emit wavelengths selected among near and short-wave infrared wavelengths; and at least one second group of pixels of the matrix comprising a photosensor sensitive to said wavelengths selected among near and short-wave infrared wavelengths.

According to one embodiment, said first and second groups form a same group.

According to one embodiment, the light emitter of the pixels of the first group is configured to emit light in the near infrared range and in the short-wave range.

According to one embodiment, the light emitter of the pixels of the first group is configured to further emit light in the visible light range.

According to one embodiment, the photosensor of pixels of a third group of pixels, belonging to the first group, is configured to sense visible light wavelengths among green, red and blue.

According to one embodiment, the photosensor of pixels of a fourth group of pixels, belonging to the first group, is configured to sense wavelengths in the near infrared range.

According to one embodiment: the photosensor of pixels of a fifth group of pixels, belonging to the first group, is configured to sense a first set of short-wave infrared wavelengths; and the photosensor of pixels of a sixth group of pixels, belonging to the first group, is configured to sense a second set of short-wave infrared wavelengths different from the first set.

According to one embodiment, the photosensor of the pixels of the second group is configured to be sensitive in the near infrared range.

According to one embodiment, the light emitter of the pixels of a seventh group of pixels, belonging to the second group, is configured to emit light in the near infrared range.

According to one embodiment, the photosensor of the pixels of the second group is configured to further be sensitive in the visible range.

According to one embodiment, the light emitter of the pixels of an eight group of pixels, belonging to the second group, is configured to emit light among green, red and blue.

According to one embodiment, the light emitter of the pixels of a ninth group of pixels, belonging to the second group, is configured to emit short-wave infrared wavelengths, or configured to emit a third set of short-wave infrared wavelengths and a fourth set of short-wave infrared wavelengths different from the third set.

According to one embodiment, each pixel comprises several light emitters or wherein the light emitters are among VCSELS, microLEDs, and microLEDs covered by a quantum dot conversion layer, or wherein the photosensors are among InGaAs photodiodes and photodiodes comprising quantum dots.

According to one embodiment, the light emitters of different pixels of the matrix emit light sequentially.

According to one embodiment, the device is a smartwatch.

### BRIEF DESCRIPTION OF THE DRAWINGS

The foregoing features and advantages, as well as others, will be described in detail in the following description of specific embodiments given by way of illustration and not limitation with reference to the accompanying drawings, in which:

FIG. 1 illustrates an example of a wearable device;

FIG. 2 illustrates a circuit of the device of FIG. 1;

FIG. 3 illustrates a circuit of the device of FIG. 1; and

FIG. 4 illustrates steps of a manufacturing method of the circuit of FIGS. 2 or 3.

### DETAILED DESCRIPTION

Like features have been designated by like references in the various figures. In particular, the structural and/or functional features that are common among the various embodiments may have the same references and may dispose identical structural, dimensional and material properties.

For the sake of clarity, only the operations and elements that are useful for an understanding of the embodiments described herein have been illustrated and described in detail.

Unless indicated otherwise, when reference is made to two elements connected together, this signifies a direct connection without any intermediate elements other than conductors, and when reference is made to two elements coupled together, this signifies that these two elements can be connected or they can be coupled via one or more other elements.

In the following disclosure, unless indicated otherwise, when reference is made to absolute positional qualifiers, such as the terms "front", "back", "top", "bottom", "left", "right", etc., or to relative positional qualifiers, such as the terms "above", "below", "higher", "lower", etc., or to qualifiers of orientation, such as "horizontal", "vertical", etc., reference is made to the orientation shown in the figures.

Unless specified otherwise, the expressions "around", "approximately", “substantially” and "in the order of" signify within 10 % or 10°, and preferably within 5 % or 5°.

In the text, quantum dots can also be referred as quantum boxes.

In the text, short-wave infrared (SWIR) wavelengths are extending from 1000 nm included to 2500 nm included.

In the text, near infrared (NIR) wavelengths are extending from 780 nm to 1000 nm excluded.

In the text, visible light for the color green includes wavelengths between approximately 520 and 565 nm, visible light for the color red includes wavelengths between approximately 625 and 740 nm, and visible light for the color blue includes wavelengths between approximately 450 and 500 nm.

In the text, ultraviolet wavelengths are extending from 100 nm to 400 nm.

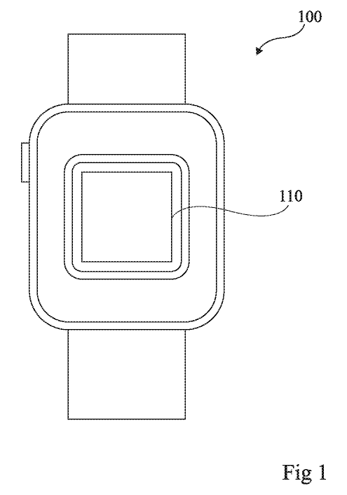

FIG. 1 illustrates an example of a wearable device 100.

The illustrated example is more particularly a backside view of a smartwatch, which is configured to be in contact with the skin of a user. This backside comprises, for example, a circuit 110 with a light emitting diode (LED), configured to emit light within the green color range (for example around 530 nm) or within the near infrared range(for example around 940 nm), and a photodiode. The light emitted by the LED is reflected by the inner structures of the user’s wrist, such as blood vessels, and the reflected light is measured by the photodiode to deduce body vital signs such as, for example, heartbeat, oximetry or blood pressure.

Visible light or near infrared are nevertheless not sufficient for accurate measurements of levels of important physiological parameters such as glucose, melanin, lactate, fat or sweat.

The LEDs and photodiodes of the current wearable devices are limited in number, typically less than ten. The current wearable devices are thus limited to a very low number of measurement points. This low accuracy is also preventing the use of the current wearable devices for biometrical applications.

The described embodiments provide a wearable device comprising an image sensor having a matrix of pixels each comprising at least one light emitter and at least one photosensor; at least one first group of pixels of the matrix comprising one light emitter configured to emit short-wave or near infrared wavelengths (SWIR, NIR); and at least one second group of pixels of the matrix comprising a photosensor sensitive to said short-wave or near infrared wavelengths (SWIR, NIR).

Such a solution allows to take accurate images across the skin and over an extended area at reasonable costs. It additionally allows an imaging across the skin without optics to create focused images.

The embodiments provided allow to record various biomarkers such as glucose, lactate, fat, water, sweat, oxygenated or deoxygenated blood, or vein imaging.

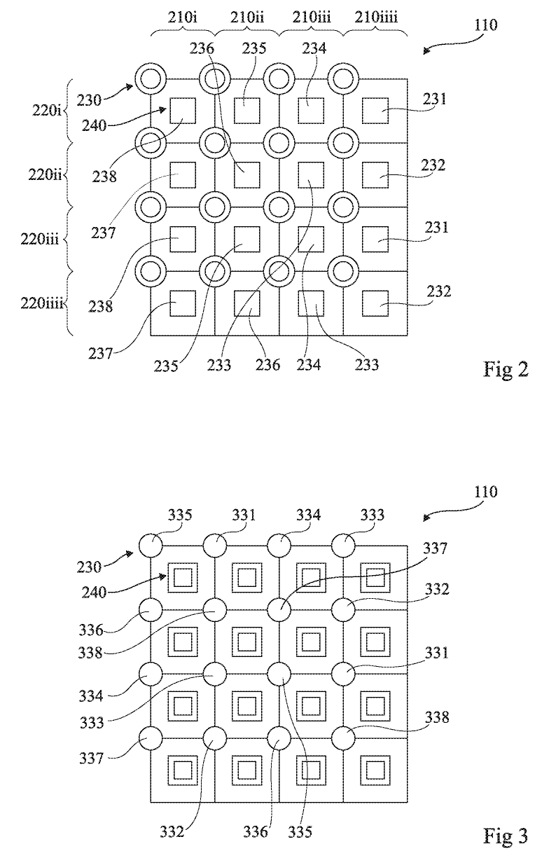

FIG. 2 illustrates an embodiment of a circuit 110 of the device 100 of FIG. 1.

In the illustrated Figure, the circuit 110 is an image sensor having a matrix of pixels arranged in rows 210(i, ii, iii, iiii) and in columns 220(i, ii, iii, iiii), further called respectively pixels 210, 220.

Each pixel 210, 220 comprises at least one light emitter 230, illustrated by a circle, and at least one photosensor 240, otherwise said a light sensor, represented by a square. In an example, each pixel comprises several light emitters. The image sensor 110 is, in an example, a monolithic array of pixels. In an embodiment, the each light emitter is positioned at a corner shared by four pixels of the matrix and the included at least one photosensor of the pixel is centrally positioned with the pixel of the matrix.

In an example, driving circuits are coupled to the pixels to drive them and/or to treat the measured data. This circuits can be arranged in between the pixels or across the pixels or under the pixels (in a manner known by those skilled in the art).

In the example of FIG. 2, all the pixels or a group of the pixels have their light emitters 230, configured to emit, for example, at least NIR or SWIR light or a broad range of wavelengths extending from visible to NIR or SWIR. In an example, a group of pixels have their emitters configured to emit a set of short-wave infrared wavelengths and another group of pixels have their emitters configured to emit another and different set of short-wave infrared wavelengths. For example, the first set is 1000 nm included to 1700 nm excluded, and the second set is from 1700 nm included to 2500 nm included.

In an example, all the pixels or some of the pixels of the group of pixels emitting SWIR wavelengths have light emitters configured to further emit light in the near infrared (NIR) range. In another example, all the pixels or some of the pixels of this same group of pixels have light emitters configured to further emit light in the visible light range, for example with wavelengths within green, red and blue domains. In a further example, the light emitters of all the pixels or some of the pixels of this same group of pixels are configured to further emit light in the ultraviolet domain.

By combining several light domains among SWIR, NIR, visible and UV, a so-called white light emitter can be achieved.

In an example, all the pixels of the matrix have the same light emitting spectrum.

In the represented example, the photosensors of a group of pixels 231, 235, 237 are respectively configured to sense wavelengths among green, red, blue or any color of the visible range, for example with narrow band selectivity (less than 100 nm of bandwidth). The photosensors of another group of pixels 232 are configured to sense wavelengths in the near infrared range. Optionally, the photosensors of another group of pixels 233 are configured to sense wavelengths in the ultraviolet range.

In an example, the photosensors of a group of pixels 234, 238 are configured to sense a first set of short-wave infrared wavelengths and the photosensors of another and different group of pixels 236 are configured to sense a second set of short-wave infrared wavelengths different from the first set. Different SWIR wavelengths channels are thus created. In other examples, the number of different sets of short-wave infrared wavelengths can be above two, for example, three, or four, or even above ten. The more channels, the better accuracy. Additionally, multiple channels allow a reinforced anti-spoofing.

In an example, different wavelengths channels can also be created in the visible, NIR or ultraviolet domains.

The light emitters 230 are, for example, vertical cavity surface emitting lasers (VCSELS), microLEDs, or microLEDs covered by a quantum dot conversion layer. MicroLEDs are, for example, made of group III-V materials such as In, Ga, As, Al, InGaAs, InGaN, and their nitride. In an example, microLEDs covered by a quantum dot conversion layer are blue GaN microLEDs.

The light sensors of groups 231, 232, 233, 234, 235, 236, 237, 238 are, for example, InGaAs photodiodes or photodiodes comprising quantum dots or photodiodes made of silicon.

A quantum dot, in the colloidal form, comprises a semiconductor core. A quantum dot may also comprise a shell, preferably of a semiconductor material, surrounding the core to protect and passivate the core. A quantum dot further comprises ligands, organic aliphatic compounds, organometallic or inorganic molecules that extend from the shell and passivate, protect and functionalize the semiconductor surface.

The composition of a quantum dot can be chosen from the following materials. For example, the core is made of one of the following materials or an alloy of the following materials: CdSe, CdS, CdTe, CdSeS, CdTeSe, AgS, ZnO, ZnS, ZnSe, CuInS, CuInSe, CuInGaS, CuInGaSe, PbS, PbSe, PbSeS, PbTe, InAsSb, InAs, InSb, InGaAs, InP, InGaP, InAlP, InGaAlP, InZnS, InZnSe, InZnSeS, HgTe, HgSe, HgSeTe, Ge, Si. The envelope is, for example, made of one of the following materials or an alloy of the following materials: CdSe, CdS, CdTe, CdSeS, CdTeSe, AgS, ZnO, ZnS, ZnSe, CuInS, CuInSe, CuInGaS, CuInGaSe, PbS, PbSe, PbSeS, PbTe, InAsSb, InAs, InSb, InGaAs, InP, InGaP, InAlP, InGaAlP, InZnS, InZnSe, InZnSeS, HgTe, HgSe, HgSeTe, Ge, Si.

Preferably, all core dimensions are smaller than 20 nm, for example in the range from 2 to 15 nm. In particular, the diameter of each quantum dot is preferably in the range from 2 to 15 nm. By diameter is meant the diameter of the smallest sphere in which the quantum dot can be inscribed.

It is possible to choose a size and dimension of quantum dots capable of absorbing, with significant absorption, any wavelength in a wide range of wavelengths. For example, it is possible to find a size and dimension of quantum dots having an operating wavelength greater than 1 µm, for example between 1 µm and 3 µm, which includes the near infrared and short infrared. For example, the light sensors of groups 231, 232, 233, 234, 235, 236, 237, 238 can be photodiodes comprising quantum dots made of InAs, PbS, HgTe or PbSe, with a radius of less than 10 nm, for example, to obtain an excitonic absorption peak in the short infrared, e.g., 1130 nm or 1360 nm.

Even the example of FIG. 2 shows a square of 4 by 4 pixels, in reality the image sensor formed may contain hundreds or thousands of pixels.

An advantage of the example of FIG. 2 is that simultaneous measurements can be achieved for different light wavelength thus having access to different physiological parameters in parallel.

Another advantage is that it forms an image sensor which provides an image of the vein structure under the skin. This advantage allows the device to discriminate spectral signatures from blood in vessels from spectral signatures from tissues surrounding the vessels, enabling to improve the detectability of chemical species, such as glucose.

Contrary to point measurements achieved by existing wearables, the use of microLEDs and micron size photodiodes in an image sensor enhance the resolution by extending drastically the number of measurement sites to form images and extending the area measured.

FIG. 3 illustrates an embodiment of a circuit of the device 100 of FIG. 1. More particularly, the represented example is an embodiment of the image sensor 110.

The example of FIG. 3 is similar to the example of FIG. 2 except that the light emitters 230 and the light sensors 240 are different.

In the example of FIG. 3, the photosensors 240, otherwise said the light sensors, are sensitive at least to short-wave or near infrared wavelengths with broadband absorption (>500 nm). In an example, the photosensors are further sensitive in the near infrared range. In another example, the photosensors are further sensitive to wavelength in the visible range such as green, red, blue or other visible colors. In a further example, the photosensors can be moreover sensitive to ultraviolet wavelengths. By combining sensitivity to several of light domains among SWIR, NIR, visible and UV, similar or identical photosensors can be used for all pixels of the matrix even if the light emitters differs from one pixel to another.

In an example, the photosensors of the matrix are photodiodes without filter.

In the represented example, the light emitters of a group 331 of pixels are configured to have narrow emission bandwidth (>100 nm) and to emit in a first SWIR wavelength domain, light emitters of another group 332 of pixels are configured to emit in a second SWIR wavelength domain, and light emitters of another group 338 of pixels are configured to emit in a second SWIR wavelength domain to create different SWIR channels. In this example, light emitters of a group 333 of pixels are configured to emit wavelengths in the visible range such as green, light emitters of a group 334 of pixels are configured to emit wavelengths in the visible range such as red, light emitters of a group 335 of pixels are configured to emit wavelengths in the visible range such as blue or other visible colors, light emitters of a group 336 of pixels are configured to emit near infrared wavelengths, and light emitters of a group 337 of pixels are configured to emit UV wavelengths.

In another example, additional channels can be envisaged in the visible or NIR or UV domains.

In an example, all pixels have light emitters configured to emit SWIR wavelengths.

In an example, not all the groups of pixels 332, 334, 335, 336, 337, 338 are implemented, and only one or several of these groups of pixels 332, 334, 335, 336, 337, 338 are implemented.

The example of FIG. 3 presents an increase in measuring points, less noise and more sensitivity.

In a functioning method of the examples of FIGS. 2 or 3, one or several images of the veins patterns are formed by emitting light, SWIR light for example, or by switching different wavelength domains, with the light emitters of the pixels, and by receiving the corresponding vein reflection with the respective photosensors. After a step of normalization, the vein structure is extracted from the image(s) treatment. Optionally, the results are encrypted and matching and/or recognition algorithms are run, for example by a computing unit of the wearable device. According to the matching and/or recognition results, the user vein pattern is recognized or not recognized. If recognized, secured operations can be enabled for example.

In another functioning method of the examples of FIGS. 2 or 3 , light is emitted by one group after another, sequentially. For example, the light emitters of group 233 emits first, then light emitters of another group for example group 234 and so on. In another example, the light emitters of group 338 emits first, then light emitters of another group, for example group 336, and so on.

By emitting light with different groups of emitters sequentially or at the same time, vital signs can be monitored, such as glucose or PPG for example. For example, heartrate can be measured by using the green pixels, oximetry can be measured by associating red and NIR pixels, breathing and blood pressure can be measured by associating green, red and NIR pixels. By using SWIR pixels, glucose, water, sweat, fat, lactate and melanin content can be detected and measured. Another of SWIR imaging is that deep veins can be reached due to a lower absorption by other under skin compounds.

Another advantage of the examples of FIGS. 2 and 3, comes from the fact that the vein pattern is detected by reflection and not by transmission which reduces drastically the footprint in volume of the measuring device and allows the integration into a wearable device. In other words, the device of FIG. 2 and 3 can be assimilated to a two-dimension wearable device as regards transmission devices which can be considered as non-wearable three-dimensional devices.

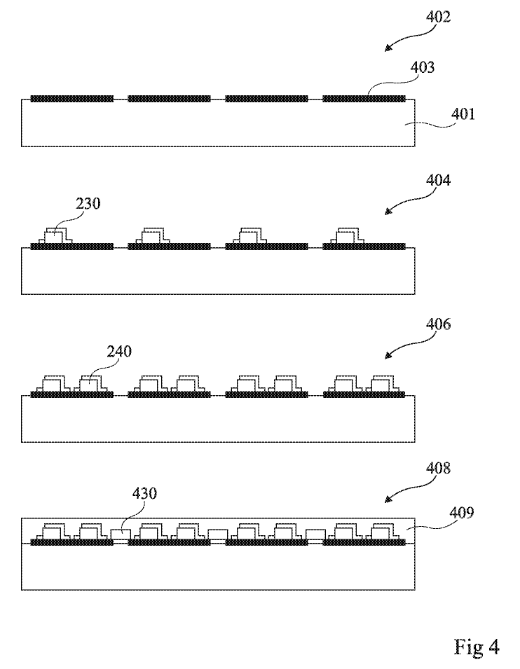

FIG. 4 illustrates steps in a manufacturing method of the circuit 110 of FIGS. 2 or 3 .

In a first step 402, power or data lines or pads 403, in a conductive material, such as metal, are formed on a flexible or solid substrate 401 made, for example, of glass or plastic, or of a metal foil.

In a second step 404, following step 402, the light emitters 230 of the pixels are mass transferred to substrate with the desired pitch. This step can be reproduced for each type of light emitter to transfer. In the case where the light emitters are microLEDs, prior to transfer, they can be manufactured on wafers with desired pitch and size.

In a third step 406, following step 404, the photosensors 240 can be, in the case of quantum dot sensors, directly formed onto the metal lines of the substrate, or in the case of silicon or III-V micro photodiodes, be mass transferred at desired pitch.

In a fourth step 408, following step 406, micro integrated circuits 430, comprising for example digital or analog drivers, read-out integrated circuit of the light emitters and/or the photosensors, are mass transferred onto the metal lines at desired pitch. Afterwards, the different elements are encapsulated with an insulating layer 409 which is further planarized.

The monolithic uLED/uPD array combo is realized using pick-and-place or mass transfer technology and processes and can be done on low-cost glass substrates or flexible substrates such as plastic or foil.

Various embodiments and variants have been described. Those skilled in the art will understand that certain features of these embodiments can be combined and other variants will readily occur to those skilled in the art. In particular, the circuit 110 of FIGS. 2 or 3 can be integrated in the back of a watch for vein imaging or the wrist for biometric identification of the owner of the watch. It can also be integrated into smart ring structures, ear sets, or any display technology as a way to do biometric identification or biomedical vital sign monitoring.

Finally, the practical implementation of the embodiments and variants described herein is within the capabilities of those skilled in the art based on the functional description provided hereinabove. In particular, the matrix of pixel can be made to have any desired pitch density (very high resolution or low resolution) of emitters and sensors over any sized area (mm² to cm² to above ten cm²) and any area shape (e.g., circle, triangle, square, rectangle).

Different applications of the veins pattern recognition allowed by the embodiments of FIGS. 2 and 3, are payment authentication, ownership certificates, data encryption, digital wallets, identity, health, passports, unlocking of doors of houses or cars.

1. A wearable device, comprising:
an image sensor having a matrix of pixels arranged in rows and columns;
wherein each pixel comprises at least one light emitter and at least one photosensor;
wherein at least one first group of pixels of the matrix comprises one light emitter configured to emit wavelengths selected among near and short-wave infrared wavelengths; and
wherein at least one second group of pixels of the matrix comprises a photosensor sensitive to said wavelengths selected among near and short-wave infrared wavelengths.

2. The device according to claim 1, wherein said first and second groups form a same group.

3. The device according to claim 1, wherein the at least one light emitter of the pixels of the first group is configured to emit light in the near infrared range and in the short-wave range.

4. The device according to claim 3, wherein the photosensor of pixels of a fourth group of pixels, belonging to the first group, is configured to sense wavelengths in the near infrared range.

5. The device according to claim 1, wherein the at least one light emitter of the pixels of the first group is configured to further emit light in the visible light range.

6. The device according to claim 1, wherein the photosensor of pixels of a third group of pixels, belonging to the first group, is configured to sense visible light wavelengths among green, red and blue.

7. The device according to claim 1, wherein:
the photosensor of pixels of a fifth group of pixels, belonging to the first group, is configured to sense a first set of short-wave infrared wavelengths; and
the photosensor of pixels of a sixth group of pixels, belonging to the first group, is configured to sense a second set of short-wave infrared wavelengths different from the first set.

8. The device according to claim 1, wherein the photosensor of the pixels of the second group is configured to be sensitive in the near infrared range.

9. The device according to claim 8, wherein the light emitter of the pixels of a seventh group of pixels, belonging to the second group, is configured to emit light in the near infrared range.

10. The device according to claim 1, wherein the photosensor of the pixels of the second group is configured to further be sensitive in the visible range.

11. The device according to claim 1, wherein the light emitter of the pixels of an eighth group of pixels, belonging to the second group, is configured to emit visible light among green, red and blue.

12. The device according to claim 1, wherein the light emitter of the pixels of a ninth group of pixels, belonging to the second group, is configured to emit short-wave infrared wavelengths, or configured to emit a third set of short-wave infrared wavelengths and a fourth set of short-wave infrared wavelengths different from the third set.

13. The device according to claim 1, wherein each pixel comprises several light emitters or wherein the light emitters are among VCSELS, microLEDs, and microLEDs covered by a quantum dot conversion layer, or wherein the photosensors are among InGaAs photodiodes and photodiodes comprising quantum dots.

14. The device according to claim 1, wherein the light emitters of different pixels of the matrix are controlled to emit light sequentially.

15. The device according to claim 1, implemented as a smartwatch.

16. The device according to claim 1, wherein the at least one light emitter of the pixel is positioned at a corner shared by four pixels of the matrix and wherein the at least one photosensor of the pixel is centrally positioned with the pixel of the matrix.

17. A method of manufacturing a device, comprising the steps of:
forming power lines, data lines and pads, made of a conductive material, on a flexible or solid substrate made of glass or plastic;
mass transferring of light emitters of pixels to the substrate with a desired pitch, where the step of mass transferring is repeated for each type of light emitter to transfer;
mass transferring of photosensors onto lines of the substrate at a desired pitch;
mass transferring micro integrated circuits onto lines of the substrate at a desired pitch;
encapsulating the light emitters, photosensors and micro integrated circuits within an insulating layer; and
planarizing the insulating layer.

18. The method according to claim 17, wherein the micro integrated circuits comprise digital or analog driver circuits for the light emitters and read-out integrated circuits for the photosensors.
